(19) United States
(12) Patent Application Publication
(10) Pub. No.: US 2024/0100116 A1
(43) Pub. Date: Mar. 28, 2024

(54) EUCOMMIA ULMOIDES OLIV. LEAF EXTRACT CAPABLE OF PROTECTING HEALTH OF KIDNEY FUNCTION, AND PREPARATION AND DETECTION METHODS THEREOF

(71) Applicants: Chenland Nutritionals Inc., Irvine, CA (US); Qingdao Chenland Health Industry Group Co., Ltd., Qingdao (CN)

(72) Inventors: Shengcan Zou, Qingdao (CN); Jinli Liu, Qingdao (CN); Weixia Wang, Qingdao (CN); Dongli Yin, Qingdao (CN); Haijun Zhang, Qingdao (CN); Decui Yin, Qingdao (CN); Shuang Feng, Qingdao (CN)

(21) Appl. No.: 18/517,494

(22) Filed: Nov. 22, 2023

(30) Foreign Application Priority Data
Aug. 31, 2023 (CN) 202311116711.0

(51) Int. Cl. A61K 36/46; A61P 13/12; G01N 30/14; G01N 30/36

(52) U.S. Cl. CPC A61K 36/46; A61P 13/12; G01N 30/14; G01N 30/36; G01N 2030/027

(57) ABSTRACT

The present invention belongs to the field of drug preparation and detection, and particularly discloses a preparation method for a Eucommia ulmoides Oliv. leaf extract. The extract is obtained from Eucommia ulmoides Oliv. leaves (the mixture of the Eucommia ulmoides Oliv. leaves and Eucommia ulmoides Oliv. male flower) by enzymolysis and extraction of ethanol and water, and can effectively solve the problem that the extract is not easy to spray dry (the Eucommia ulmoides Oliv. leaves contain colloidal substances). The pharmacodynamic experiment proves that the Eucommia ulmoides Oliv. leaf extract has good effect of protecting the health of the renal function. The present invention also develops a detection method for the extract, which not only realizes the quality control of the extract, but also has the advantages of good repeatability and stability.

### TECHNICAL FIELD

The present invention belongs to the field of drug preparation and detection, and particularly relates to a Eucommia ulmoides oliv. leaf extract capable of protecting health of kidney function, and preparation and detection methods thereof.

### BACKGROUND

Eucommia ulmoides Oliv. leaves are dry leaves of Eucommia ulmoides Oliv. as a plant in Eucommiaceae, and can strengthen the muscles and bones and nourish liver and kidney. Pharmacopoeia of the People's Republic of China, Volume I, 2005 edition has officially included Eucommia ulmoides Oliv. leaves in the legal standard. The research indicates that Eucommia ulmoides Oliv. leaves and barks have similar chemical composition and pharmacological effects.

The Eucommia ulmoides Oliv. leaves are rich in various active components which are mainly divided into the following categories: flavonoid compounds mainly include quercetin, kaempferide, rutin, astragalin, isoquercitrin, hyperoside, kaempferol, quercitrin, etc. The main components of the iridoid compound include aucubin, geniposidic acid, geniposide, eucommiol, eucommioside and genipin. Common polyphenol compounds mainly include caffeic acid, chlorogenic acid, protocatechuic acid, syringin, laricinolic acid, coniferin and coumaric acid, wherein the Eucommia ulmoides Oliv. leaves are rich in chlorogenic acid, which is higher than that of the barks and male flower, and the content is 3%-5%. Eucommia ulmoides Oliv. polysaccharides are active components found in Eucommia ulmoides Oliv. leaves in recent years. Relevant research proves that the content of total sugar in the Eucommia ulmoides Oliv. leaves is about 30%, and the main biological activities are antioxidation, anticomplement, antifatigue, immuno-enhancement and antitumor. In addition, the Eucommia ulmoides Oliv. leaves also have significant effects on the treatment of osteoporosis, diabetes and “hyperglycemia, hypertension and hyperlipemia”, have extremely high values in protecting cardiovascular system, liver and kidney, and vision, and has no toxic or side effect.

Traditionally, the bark of Eucommia ulmoides Oliv. is the main medicine, and now the Eucommia ulmoides Oliv. leaves can also be used as medicine. Therefore, the Eucommia ulmoides Oliv. leaves with homology of medicine and food are the good high-quality raw materials for the development of modern Chinese medicine, health care products and functional food and drinks.

At present, the quality of the Eucommia ulmoides Oliv. leaf extracts on the market is different. Most of the Eucommia ulmoides Oliv. leaf extracts are based on water extraction technology, and have simple standard indexes, mainly for detection of chlorogenic acid content; and the analysis of other active components in the extracts is not clear.

### SUMMARY

In view of this, the first purpose of the present invention is to provide a preparation method for a Eucommia ulmoides Oliv. leaf extract with respect to the problems existing in the prior art, which can not only increase the yield and improve the fluidity of the spray-dried powder of the traditional extract, but also clarify the characteristic peak components in the fingerprint of the extract and increase the content of some beneficial substances (quercetin, kaempferol, etc.).

It should be noted that the current extraction technologies of the Eucommia ulmoides Oliv. leaves are still mainly traditional ethanol extraction and water extraction. The present invention uses the complex enzyme extraction technology to achieve the transformation of some components with specific effects, and develops a fingerprint detection method of the corresponding product while improving the yield, which can conduct multi-index qualitative and quantitative analysis of the extracted products and can effectively control the quality stability of different batches of products.

Moreover, at present, there is almost no extract product of Eucommia ulmoides Oliv. leaves (or compound medicine of Eucommia ulmoides Oliv. leaves and Eucommia ulmoides Oliv. male flower) on the market. The present invention makes deep development and research on the enzymolysis and extraction technologies of the Eucommia ulmoides Oliv. leaves or the compound medicine of the Eucommia ulmoides Oliv. leaves and the Eucommia ulmoides Oliv. male flower, verifies the drug effects in vivo and in vitro, and fills the gaps in domestic and foreign markets to a certain extent.

To achieve the above purpose, the present invention adopts the following technical solution:
A preparation method for a Eucommia ulmoides Oliv. leaf extract comprises the following steps:
1) soaking the coarse powder of Eucommia ulmoides Oliv. leaves or mixed coarse powder of Eucommia ulmoides Oliv. leaves and male flower in water, and then adding a complex enzyme for enzymolysis and filtration to obtain drug residues and extracting solution I;
2) adding ethanol to the drug residues prepared in step 1) for extracting for 1-2 times; and after screening and filtering, obtaining drug residues and extracting solution II;
3) adding water to the drug residues prepared in step 2), and after extracting for 1-2 times, filtering to obtain extracting solution III;
4) concentrating the extracting solution I, the extracting solution II and the extracting solution III respectively; cooling the concentrates to room temperature and then mixing the concentrates; standing and then filtering; and spray-drying the filtrate to obtain the Eucommia ulmoides Oliv. leaf extract.

It is worth noting that, due to the similar effects of Eucommia ulmoides Oliv. male flower and Eucommia ulmoides Oliv. leaf components, when the Eucommia ulmoides Oliv. male flower is added to the Eucommia ulmoides Oliv. leaves according to different proportions, the state of the material after spray drying can be improved. Meanwhile, the enzymolysis step is conducive to reducing the interference of Eucommia rubber on the spray drying process, so that the extract has better fluidity and the yield is increased.

Optionally, the material-solution ratio of the coarse powder or mixed coarse powder of the Eucommia ulmoides Oliv. leaves to the water is 1:10-20, and the complex enzyme is a mixture of cellulase and pectinase with a mass ratio of 1:1-3:1; the addition amount of the complex enzyme is 1%-2% of the mass of crude drug, enzymolysis time is 1-3 hours, and enzymolysis temperature is 45° C.-50° C.

Optionally, in step 2), the material-solution ratio of the drug residues to the ethanol is 1:6-10, the ethanol concentration is 70%-80%, and the extraction time is 1-3 hours each time.

Optionally, in step 3), the material-solution ratio of the drug residues to the water is 1:6-1:8, and the extraction time is 1-2 hours each time.

Optionally, the concentration technology in the step 4) is rotary evaporation with a vacuum degree of −0.06 to −1 Mpa, temperature of 60-80° C. and speed of 40-60 rpm; the density of the extract at 25° C. is 1.12-1.14; and the technological parameters for centrifugal spray drying of the obtained concentrates are as follows:
inlet air temperature is 150-155° C., outlet air temperature is 120-125° C., the centrifugal frequency is 280 Hz, induced air frequency is 50 Hz, and the speed of a feed pump is 20 r/min.

Further, the drugs are soaked in water; the material-solution ratio is 1:10; 1% complex enzyme is added; enzymolysis is conducted for 2 h at enzymolysis temperature of 50° C.; the material-solution ratio of the drug residues to the ethanol for extracting is 1:8, the ethanol concentration is 75%; the extraction times are 1, and the extraction time is 2 hours; the material-solution ratio of the drug residues to the water for extracting is 1:6, and the extraction times are 2, with 2 hours for each time; and after the two extracting solutions are screened and filtered through a 60-80-mesh sieve, the extracting solutions are merged and concentrated, and the density of the concentrated extract is 1.13 (25° C.).

The second purpose of the present invention is to provide a Eucommia ulmoides Oliv. leaf extract prepared by the above method.

The Eucommia ulmoides Oliv. leaf extract is obtained from the Eucommia ulmoides Oliv. leaves or the mixture of the Eucommia ulmoides Oliv. leaves and the Eucommia ulmoides Oliv. male flower by enzymolysis and complex extraction of ethanol and water; and the mass percentages of the Eucommia ulmoides Oliv. leaves and the Eucommia ulmoides Oliv. male flower in the mixture are as follows:
80%-95% of the Eucommia ulmoides Oliv. leaves and 5%-20% of the Eucommia ulmoides Oliv. male flower;
and the Eucommia ulmoides Oliv. leaf extract has the effect of protecting the health of the kidney function.

Further, the mass ratio of the Eucommia ulmoides Oliv. leaves to the Eucommia ulmoides Oliv. male flower is 90:10.

The third purpose of the present invention is to provide a fingerprint detection method for the Eucommia ulmoides Oliv. leaf extract.

A high performance liquid chromatography is used for constructing a chromatogram, and chromatographic conditions are as follows:
chromatographic columns use octadecyl silane chemically bonded to porous silica as fillers; column temperature is 25-35° C.; a flow rate is 0.9-1.1 mL/min; a detection wavelength is 203-337 nm; a mobile phase A is 0.4% phosphate water, and a mobile phase B is acetonitrile; and a gradient elution procedure is:

| | Mobile | Mobile |
|---|---|---|
| Time (min) | phase A (%) | phase B (%) |
| 0 | 95 | 5 |
| 10 | 90 | 10 |
| 30 | 88 | 12 |
| 65 | 70 | 30 |
| 85 | 5 | 95 |
| 86 | 95 | 5 |

Further, the detection wavelength is 254 nm, the chromatographic columns are Shim-pack VP-ODS, and the specification is: 4.6×250 mm, 5 μm; the column temperature is 35° C.; and the flow rate is 1.0 mL/min.

Optionally, for a test solution used for liquid chromatography determination in the method, a preparation method thereof comprises:
accurately weighing 0.5 g of Eucommia ulmoides Oliv. leaf complex extract in a 100 ml conical flask, adding 25 mL of 50% methanol water, sealing by ultrasound at room temperature for 30 min, shaking well, and filtering to obtain the test solution.

In addition, a preparation method for a reference solution comprises: preparing various reference substances used for fingerprint detection into 1 mg/mL reference solutions with methanol respectively, blending 1 mL respectively and filtering to obtain the mixed reference solution.

Compared with the prior art, the present invention has the following excellent effects:
1) The Eucommia ulmoides Oliv. leaf extract disclosed by the present invention is obtained from the Eucommia ulmoides Oliv. leaves (the mixture of the Eucommia ulmoides Oliv. leaves and the Eucommia ulmoides Oliv. male flower) by enzymolysis and extraction of ethanol and water, and can effectively solve the problem that the extract is not easy to spray dry (the Eucommia ulmoides Oliv. leaves contain colloidal substances). The pharmacodynamic experiment proves that the Eucommia ulmoides Oliv. leaf extract has good effect of protecting the health of the renal function.
2) The present invention develops a detection method for the extract, which can be used for qualitative and quantitative analysis of the main active substances (such as chlorogenic acid, geniposidic acid, quercetin, etc.) in the Eucommia ulmoides Oliv. leaf extract. At the same time, it is found that the fingerprint of the sample after enzymolysis shows that the content of quercetin and kaempferol is higher than that of the sample without enzymolysis, and beneficial transformation of some components may occur in the process of enzymolysis. The development of the fingerprint detection method can find the quality difference between product batches in time to ensure the stability of products and drug efficacy.

### DESCRIPTION OF DRAWINGS

To more clearly describe the technical solutions in the embodiments of the present invention or in the prior art, the drawings required to be used in the description of the embodiments or the prior art will be simply presented below. Apparently, the drawings in the following description are merely the embodiments of the present invention, and for those ordinary skilled in the art, other drawings can also be obtained according to the provided drawings without contributing creative labor.

FIG. 1 is a flow chart of a preparation technology of a Eucommia ulmoides Oliv. leaf extract in the present invention.

FIG. 2 is a diagram of the inhibitory effect of allopurinol on xanthine oxidase.

FIG. 3 shows the inhibitory effect of the Eucommia ulmoides Oliv. leaf extract on xanthine oxidase.

FIG. 4 shows effect diagrams of the Eucommia ulmoides Oliv. leaf extract on UR FIG. 4(A), UA FIG. 4(B), CR FIG. 4(C), cystatin C FIG. 4(D), complement C1q FIG. 4(E) and urine protein FIG. 4 (F) in serum of adenine-induced CKD rats.

FIG. 5 shows MDA FIG. 5(A), T-AOC FIG. 5(B), TNF-α FIG. 5(C) and 1L-1β FIG. 5(D) diagrams of antioxidant and anti-inflammatory effects of the Eucommia ulmoides Oliv. leaf extract on CKD rats.

FIG. 6 shows influence diagrams of the Eucommia ulmoides Oliv. leaf extract on renal histopathology in rats, wherein FIG. 6(A) is a blank group, FIG. 6(B) is a model group, FIG. 6(C) is an allopurinol group, FIG. 6(D) is the Eucommia ulmoides Oliv. leaf extract, and FIG. 6(E) is the composite extract of Eucommia ulmoides Oliv. leaves and male flower.

FIG. 7 shows fingerprints of the Eucommia ulmoides Oliv. leaf extract, wherein FIG. 7(A) is a control standard, FIG. 7(B) is a sample directly extracted without enzymolysis process, and FIG. 7(C) is a Eucommia ulmoides Oliv. leaf sample after enzymolysis.

### DETAILED DESCRIPTION

The technical solutions in embodiments of the present invention will be clearly and fully described below. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

Any embodiment described herein by the term “embodiment” as “exemplary” need not be interpreted as superior to or better than other embodiments. Unless otherwise specified, the performance index test in the embodiments of the present application uses the conventional test methods in the field. It should be understood that the terms in the present application are only intended to describe particular embodiments and not intended to limit the disclosure of the present application.

Unless otherwise specified, the technical and scientific terms used herein have the same meanings as those generally understood by those ordinary skilled in the technical field to which the present application belongs. Other test methods and technical means not specifically indicated in the present application refer to the experiment methods and technical means generally used by those ordinary skilled in the art.

The terms “basically” and “approximately” used herein are used for describing small fluctuation. For example, they can mean less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. Numerical data represented or presented in the range format herein is used for convenience and brevity only and should therefore be interpreted flexibly to include not only the numerical values explicitly enumerated as the limits of the range, but also all independent numerical values or subranges contained within the range. For example, the numerical range of “1 to 5%” should be interpreted to include not only the explicitly enumerated values of 1% to 5%, but also independent values and subranges within the shown range. Therefore, this numerical range includes independent values such as 2%, 3.5% and 4%, and subranges such as 1%-3%, 2%-4% and 3%-5%. This principle also applies to ranges that list only one numerical value. In addition, such interpretation applies regardless of the width of the range or the features.

To better explain the content of the present application, numerous specific details are given in the specific embodiments below. Those skilled in the art should understand that the present application can be implemented without certain specific details. In embodiments, some methods, means, instruments, equipment, etc. well known to those skilled in the art are not described in detail in order to highlight the purport of the present application.

On the premise of no conflict, the technical features disclosed in the embodiments of the present application can be arbitrarily combined, and the obtained technical solution belongs to the disclosure of the embodiments of the present application.

To better understand the present invention, the present invention is further described in detail below by the following embodiments, but shall not be interpreted as a limitation to the present invention. Some non-essential improvements and adjustments made by those skilled in the art according to the contents of the present invention shall also be deemed to fall within the protection scope of the present invention.

### Embodiment 1

### Research on Enzymolysis Extraction Technology of Eucommia ulmoides Oliv. Leaves

### 1) Enzyme Proportion Experiment

A certain amount of coarse powder of Eucommia ulmoides Oliv. leaves is divided equally into 3 groups; each group is soaked for 1 h with 10 times the amount of water; 1% complex enzyme (cellulase:pectinase w:w: is 2:1) is added to group 1; 1% complex enzyme (cellulase:pectinase w:w: is 1:1) is added to group 2; 1% complex enzyme (cellulase:pectinase W:W: is 1:2) is added to group 3; stirring is conducted for enzymolysis at 50° C. for 2 h; and the enzymolysis solution is filtered out. The remaining drug residues of the 3 groups are extracted for 2 h according to the 75% ethanol concentration of the extracting solution at 8 times the amount; the ethanol extract is filtered out; the drug residues are continuously extracted with water twice; dosages are 8 times and 6 times respectively; the extraction time is 2 h/time; and water extracts are merged twice. In the above process, the enzymolysis solution, the ethanol extract and the water extract are concentrated, dried under reduced pressure respectively, and crushed through an 80-mesh sieve. Three samples, i.e., sample 1, sample 2 and sample 3 are obtained respectively in the three groups.

| Enzymolysis | Chlorogenic acid | |
|---|---|---|
| product | transfer rate (%) | Yield (%) |
| Sample 1 | 78.42 | 41.56% |
| Sample 2 | 82.72 | 42.28% |
| Sample 3 | 76.14 | 42.72% |

It can be seen from the above results that the chlorogenic acid transfer rate of sample 2 is the highest, the yields of sample 2 and sample 3 are slightly higher than that of sample 1, and there is no significant difference between sample 2 and sample 3. Therefore, cellulase:pectinase w:w of 1:1 is preferably selected as the enzyme proportion.

### 2) Enzyme Addition Amount Experiment

Enzyme preparations used are all complex enzymes with cellulase:pectinase w:w of 1:1.

A certain amount of coarse powder of Eucommia ulmoides Oliv. leaves is divided equally into 4 groups; each group is soaked for 1 h with 10 times the amount of water; complex enzymes with dosages of 0.5%, 1%, 2% and 3% (compared with the mass of the crude drug) are added to groups 1-4 respectively; stirring is conducted for enzymolysis at 50° C. for 2 h; and the enzymolysis solution is filtered out. The remaining drug residues of the 4 groups are extracted for 2 h according to the 75% ethanol concentration of the extracting solution at 8 times the amount; the ethanol extract is filtered out; the drug residues are continuously extracted with water twice; dosages are 8 times and 6 times respectively; the extraction time is 2 h/time; and water extracts are merged twice. In the above process, the enzymolysis solution, the ethanol extract and the water extract are concentrated, dried under reduced pressure respectively, and crushed through an 80-mesh sieve. Sample 1-sample 4 are obtained respectively in the four groups.

| Enzymolysis | Chlorogenic acid | | Angle of |
|---|---|---|---|
| product | transfer rate (%) | Yield (%) | repose º |
| Sample 1 | 70.31 | 39.06% | 19.14° |
| Sample 2 | 81.64 | 41.18% | 21.40° |
| Sample 3 | 81.92 | 40.96% | 35.69° |
| Sample 4 | 80.11 | 41.13 | 40.58° |

It can be seen from the above table that the chlorogenic acid transfer rate is increased with the increase of the addition amount of the complex enzymes, but the increase rate becomes lower when the addition amount is more than 1%, and there is no significant difference. The yield of the extract reaches the highest value when the addition amount is 1%, and then the yield is almost unchanged when the amount of enzyme is increased. It can be seen from the data of the angle of repose that with the increase of the amount of the enzyme, the angle of repose is increased and the material fluidity becomes worse, which may be caused by the increase of Eucommia rubber or enzymolysis carbohydrates in the product. After comprehensive consideration, the addition amount of the complex enzyme with cellulase:pectinase w:w of 1:1 is 1%.

### Embodiment 2

### Comparison of Enzymolysis Extraction Technology and Traditional Technology of Eucommia ulmoides Oliv. Leaves

\textcircled{1} Traditional technology: a certain amount of coarse powder of Eucommia ulmoides Oliv. leaves is taken; 8 times the amount of 75% ethanol (based on the weight) is added for extracting once for 2 h; the ethanol extract is collected; 6 times the amount of aqueous solution is added to drug residues for extracting twice, for 2 h each time, and the water extract is collected; and the obtained ethanol extract and water extract are concentrated respectively. Concentration conditions: vacuum degree <−0.08 MPa, temperature: 60° C. for the ethanol extract, 80° C. for the water extract; Concentration is conducted until the specific gravity is 1.13-1.14; the ethanol extract and the water extract are mixed, restored to room temperature, stood for 1 h, and filtered with a 60-mesh sieve to remove the bottom colloidal substance; 10% crude drug of maltodextrin is added to the filtrate, blended and spray-dried centrifugally. Parameters are: inlet air temperature is 150-155° C., outlet air temperature is 120-125° C., the centrifugal frequency is 280 Hz, induced air frequency is 50 Hz, and the speed of a feed pump is 20 RPM. Finally, a spray-dried product \textcircled{1} is obtained.

\textcircled{2} Enzymolysis technology: a certain amount of coarse powder of Eucommia ulmoides Oliv. leaves is soaked in 10 times the amount of water for 1 h; 1% complex enzyme (cellulase:pectinase is 1:1) is added; stirring is conducted at 50° C. for 2 h; the solution is filtered; 8 times the amount of 75% ethanol (based on the weight) is added to the remaining drug residues for extracting once for 2 h; and the ethanol extract is collected. 6 times the amount of aqueous solution is added to drug residues for extracting twice, for 2 h each time, and the water extract is collected. The obtained enzymolysis solution, the ethanol extract and the water extract are concentrated respectively. Concentration conditions: vacuum degree <−0.08 MPa, temperature: 60° C. for the ethanol extract, and 90° C. for the water extract. Concentration is conducted until the specific gravity is 1.13-1.14; the enzymolysis solution, the ethanol extract and the water extract are mixed, restored to room temperature, stood for 1 h, and filtered with a 60-mesh sieve to remove the bottom colloidal substance; 10% crude drug of maltodextrin is added to the filtrate, blended and spray-dried centrifugally. Parameters are: inlet air temperature is 150-155° C., outlet air temperature is 120-125° C., the centrifugal frequency is 280 Hz, induced air frequency is 50 Hz, and the speed of a feed pump is 20 RPM. Finally, a spray-dried product \textcircled{2} is obtained.

The detection results of the spray-dried products are as follows:

| | | | Spray-drying | |
|---|---|---|---|---|
| Spray-dried | Spray-dried | | viscosity | Particle |
| product | product (g) | Yield | condition | size |
| \textcircled{1} | 3901 | 39.01% | Very seriously | 80.51% |
| | | | viscous | Screened by an |
| | | | | 80-mesh sieve |
| \textcircled{2} | 4328 | 43.28% | Slightly viscous | 96.30% |
| | | | | Screened by an |
| | | | | 80-mesh sieve |

| Spray-dried | Angle of | |
|---|---|---|
| product | repose ° | Fluidity determination |
| \textcircled{1} | 54.35° | Poor |
| | | (-must stir and shake) |
| \textcircled{2} | 11.31° | Very good |

It can be seen from the spray drying state of the product that when the traditional technology is used for spray drying, the product is seriously viscous and the yield is low, and when the angle of repose exceeds 50°, the fluidity of the powder is difficult to satisfy the production demand, which is not suitable for mass production.

The addition of the enzymolysis technology can not only increase the yield, but also remove the colloidal components in the Eucommia ulmoides Oliv. leaves to alleviate the viscosity phenomenon of the spray-dried powder. The finally obtained extract has an angle of repose of 11.31° and very good fluidity, and can satisfy the requirements for the fluidity of the powder in the production process.

### Embodiment 3

### Ethanol Extraction Orthogonal Experiments of Eucommia ulmoides Oliv. Leaf Extract

A certain amount of coarse powder of Eucommia ulmoides Oliv. leaves is soaked in 10 times the amount of water for 1 h; 1% complex enzyme (cellulase:pectinase w:w is 1:1) is added; stirring is conducted at 50° C. for 2 h; the solution is filtered; the remaining drug residues are based on 70% ethanol concentration, 75% ethanol concentration and 80% ethanol concentration of the extracting solution; 3-factor and 3-level orthogonal experiments are carried out according to the material-solution ratios of extraction of 1:6, 1:8 and 1:10 and extraction time of 1 h, 1.5 h and 2 h; and the content of chlorogenic acid in the extract is used as a judgment standard.

TABLE 1
Orthogonal Experiment Factors and Levels

| | Factor | | | |
|---|---|---|---|---|
| | A | B | C | |
| | Ethanol | Material-solution | Extraction | e |
| Level | concentration % | ratio | time h | Error |
| 1 | 70 | 1:6 | 1 | 1 |
| 2 | 75 | 1:8 | 1.5 | 2 |
| 3 | 80 | 1:10 | 2 | 3 |

TABLE 2
L9(34) Orthogonal Experimental Results

| | A | B | C | | |
|---|---|---|---|---|---|
| | Ethanol | Material-solution | Extraction | e | Chlorogenic acid |
| | concentration | ratio | time | Error | content % |
| Experiment 1 | 1 | 1 | 1 | 1 | 2.94 |
| Experiment 2 | 1 | 2 | 2 | 2 | 3.88 |
| Experiment 3 | 1 | 3 | 3 | 3 | 4.01 |
| Experiment 4 | 2 | 1 | 2 | 3 | 3.9 |
| Experiment 5 | 2 | 2 | 3 | 1 | 4.69 |
| Experiment 6 | 2 | 3 | 1 | 2 | 3.04 |
| Experiment 7 | 3 | 1 | 3 | 2 | 3.97 |
| Experiment 8 | 3 | 2 | 1 | 3 | 3.85 |
| Experiment 9 | 3 | 3 | 2 | 1 | 4.08 |
| Mean value 1 | 3.61 | 3.603 | 3.277 | 3.903 | |
| Mean value 2 | 3.877 | 4.14 | 3.953 | 3.63 | |
| Mean value 3 | 3.967 | 3.71 | 4.223 | 3.92 | |
| Range | 0.357 | 0.537 | 0.946 | 0.29 | |

TABLE 3
Analysis of Variance

| | | Degree of | | F critical | |
|---|---|---|---|---|---|
| Factor | | freedom | F ratio | value | Significance |
| A | Ethanol concentration | 2 | 487 | 19 | * |
| B | Material-solution ratio | 2 | 468 | 19 | * |
| C | Extraction time | 2 | 640 | 19 | * |
| e | Error | 2 | 1 | 19 | |
| | Error | 2 | | | |

The above analysis of variance indicates that the ethanol concentration, the material-solution ratio and the extraction time are significant in the extraction of the Eucommia ulmoides Oliv. leaves. Therefore, the preferred technology is A3B2C3, which conducts extraction for 2 h according to the ethanol concentration of 80% and the material-solution ratio of 1:8. However, considering the cost and safety problems of 80% ethanol, an optimized technology is extraction for 2 h according to the ethanol concentration of 75% and the material-solution ratio of 1:8.

### Embodiment 4

### Water Extraction Orthogonal Experiments of Eucommia ulmoides Oliv. Leaf Extract

The remaining drug residues after extraction in embodiment 1 are mixed evenly, and subjected to water extraction. 3-factor and 2-level orthogonal experiments are carried out according to the material-solution ratios of extraction of 1:6, 1:8 and 1:10, extraction time of 1 h, 1.5 h and 2 h and the number of times of extraction of 1, 2 and 3; and the content of chlorogenic acid in the extract is used as a judgment standard.

TABLE 4
Orthogonal Experiment Factors and Levels

| | Factor | | | |
|---|---|---|---|---|
| | A | B | C | |
| | Number of times | Material-solution | Extraction | e |
| Level | of extraction | ratio | time h | Error |
| 1 | 1 | 1:6 | 1 | 1 |
| 2 | 2 | 1:8 | 1.5 | 2 |

TABLE 5
L4(23) Orthogonal Experimental Results

| | Factor | | | |
|---|---|---|---|---|
| | A | B | | |
| | Number | Material- | C | |
| | of times | solution | Extraction | Experimental |
| Column | of extraction | ratio | time | results |
| Experiment 1 | 1 | 1:06 | 1 | 1.65 |
| Experiment 2 | 1 | 1:08 | 2 | 2.07 |
| Experiment 3 | 2 | 1:06 | 2 | 2.41 |
| Experiment 4 | 2 | 1:08 | 1 | 1.73 |

TABLE 6
Analysis of Variance

| | Sum of | | | |
|---|---|---|---|---|
| | square | Degree of | | F critical |
| Factor | of error | freedom | F ratio | value |
| Number of times of | 0.044 | 1 | 2.588 | 161 |
| extraction | | | | |
| Material-solution ratio | 0.017 | 1 | 1 | 161 |
| Extraction time | 0.303 | 1 | 17.824 | 161 |
| Error | 0.02 | 1 | | |

The above analysis of variance indicates that the preferred technology is A₂B₁C₂, which conducts extraction twice, for 2 h each time, according to the material-solution ratio of 1:6.

### Embodiment 5

### Pilot Scale Production

### I Instruments and Equipment

1. Extraction, Ethanol Precipitation and Concentration Units (Beijing Shunfu Technology Co., Ltd. SFJZ-200)
2. Centrifugal Twin-Fluid Spray Dryer (Shanghai Qiaofeng Industrial Co., Ltd. QFN-LE-5)

### II Production Technology

### 2.1 Extraction

The Eucommia ulmoides Oliv. leaves and the Eucommia ulmoides Oliv. male flower are coarsely ground respectively.

\textcircled{1} The above materials are added to an extraction tank according to the 100:0 ratio (10 kg of Eucommia ulmoides Oliv. leaves). 10 times the amount of water is added for soaking for 1 h; 1% complex enzyme (cellulase:pectinase is 1:1) is added; stirring is conducted at 50° C. for 2 h; the solution is filtered; and the enzymolysis solution is collected. 8 times the amount of 75% ethanol (based on the weight) is added to the remaining drug residues for extracting once for 2 h; and the ethanol extract is collected. 6 times the amount of aqueous solution is added to the drug residues for extracting twice, for 2 h each time, and the water extract is collected.

\textcircled{2} The above materials are added to the extraction tank according to the 90:10 ratio (9 kg of Eucommia ulmoides Oliv. leaves and 1 kg of Eucommia ulmoides Oliv. male flower). 10 times the amount of water is added for soaking for 1 h; 1% complex enzyme (cellulase:pectinase is 1:1) is added; stirring is conducted at 50° C. for 2 h; the solution is filtered; and the enzymolysis solution is collected. 8 times the amount of 75% ethanol (based on the weight) is added for extracting once for 2 h; and the ethanol extract is collected. 6 times the amount of aqueous solution is added to the drug residues for extracting twice, for 2 h each time, and the water extract is collected.

\textcircled{3} The above materials are added to the extraction tank according to the 80:20 ratio (8 kg of Eucommia ulmoides Oliv. leaves and 2 kg of Eucommia ulmoides Oliv. male flower). 10 times the amount of water is added for soaking for 1 h; 1% complex enzyme (cellulase:pectinase is 1:1) is added; stirring is conducted at 50° C. for 2 h; the solution is filtered; and the enzymolysis solution is collected. 8 times the amount of 75% ethanol (based on the weight) is added for extracting once for 2 h; and the ethanol extract is collected. 6 times the amount of aqueous solution is added to the drug residues for extracting twice, for 2 h each time, and the water extract is collected.

\textcircled{4} The above materials are added to the extraction tank according to the 50:50 ratio (5 kg of Eucommia ulmoides Oliv. leaves and 5 kg of Eucommia ulmoides Oliv. male flower). 10 times the amount of water is added for soaking for 1 h; 1% complex enzyme (cellulase:pectinase is 1:1) is added; stirring is conducted at 50° C. for 2 h; the solution is filtered; and the enzymolysis solution is collected. 8 times the amount of 75% ethanol (based on the weight) is added for extracting once for 2 h; and the ethanol extract is collected. 6 times the amount of aqueous solution is added to the drug residues for extracting twice, for 2 h each time, and the water extract is collected.

### 2.2 Concentration

The enzymolysis solution, the ethanol extract and the water extract obtained in 2.1 \textcircled{1} are concentrated respectively. Concentration conditions: vacuum degree <−0.08 MPa, temperature: 60° C. for the ethanol extract, and 90° C. for the water extract (enzymolysis). Concentration is conducted until the specific gravity is 1.13-1.14; the enzymolysis solution, the ethanol extract and the water extract are mixed, restored to room temperature, stood for 1 h, and filtered with a 60-mesh sieve to remove the bottom colloidal substance. 10% crude drug of maltodextrin is added to the filtrate and blended. Concentrate \textcircled{1} is obtained.

The enzymolysis solution, the ethanol extract and the water extract obtained in 2.1 \textcircled{2} are concentrated respectively. Concentration conditions: vacuum degree <−0.08 MPa, temperature: 60° C. for the ethanol extract, and 90° C. for the water extract (enzymolysis). Concentration is conducted until the specific gravity is 1.13-1.14; the enzymolysis solution, the ethanol extract and the water extract are mixed, restored to room temperature, stood for 1 h, and filtered with a 60-mesh sieve to remove the bottom colloidal substance. 10% crude drug of maltodextrin is added to the filtrate and blended. Concentrate \textcircled{2} is obtained.

The enzymolysis solution, the ethanol extract and the water extract obtained in 2.1 \textcircled{3} are concentrated respectively. Concentration conditions: vacuum degree <−0.08 MPa, temperature: 60° C. for the ethanol extract, and 90° C. for the water extract (enzymolysis). Concentration is conducted until the specific gravity is 1.13-1.14; the enzymolysis solution, the ethanol extract and the water extract are mixed, restored to room temperature, stood for 1 h, and filtered with a 60-mesh sieve to remove the bottom colloidal substance. 10% crude drug of maltodextrin is added to the filtrate and blended. Concentrate \textcircled{3} is obtained.

The enzymolysis solution, the ethanol extract and the water extract obtained in 2.1 \textcircled{4} are concentrated respectively. Concentration conditions: vacuum degree <−0.08 MPa, temperature: 60° C. for the ethanol extract, and 90° C. for the water extract (enzymolysis). Concentration is conducted until the specific gravity is 1.13-1.14; the enzymolysis solution, the ethanol extract and the water extract are mixed, restored to room temperature, stood for 1 h, and filtered with a 60-mesh sieve to remove the bottom colloidal substance. 10% crude drug of maltodextrin is added to the filtrate and blended. Concentrate is obtained.

### 2.3 Spray Drying

The concentrates \textcircled{1}, \textcircled{2}, \textcircled{3} and \textcircled{4} obtained in 2.2 are spray-dried centrifugally. Parameters are: inlet air temperature is 150-155° C., outlet air temperature is 120-125° C., the centrifugal frequency is 280 Hz, induced air frequency is 50 Hz, and the speed of a feed pump is 20 RPM.

\textcircled{1} The equipment is cleaned before use to satisfy the relevant requirements.

\textcircled{2} A compressor is turned on. The inlet air temperature is 160° C., the outlet air temperature is 120-125° C., and the induced air frequency is 50 Hz. When the temperature is constant and can be felt at the outer side of a spray tower wall, feeding is conducted.

\textcircled{3} The feed pump is turned on at 20 RPM and the tower is shaken every 3-5 min to prevent the materials from sticking to the tower.

\textcircled{4} After all the concentrates are sucked, a feed hole can be replaced with pure water for flushing the remaining concentrates in the feed hole.

\textcircled{5} The feed pump is turned off, and the inlet air temperature, the outlet air temperature, the induced air frequency, and the compressor are turned off.

\textcircled{6} After the temperature is recovered to room temperature, the spray drying materials in a middle tower and a large tower are taken out, mixed evenly, and screened by an 80-mesh sieve.

\textcircled{7} The equipment is disassembled and cleaned.

2.4 The detection results of the spray-dried products are as follows:

| | Obtained | | | |
|---|---|---|---|---|
| Spray-dried | spray-dried | | Spray-drying | |
| product | product (g) | Yield | viscosity condition | Particle size |
| \textcircled{1} | 4048 | 40.48% | Greatly viscous | 87.33% |
| | | | | Screened by an |
| | | | | 80-mesh sieve |
| \textcircled{2} | 4325 | 43.25% | Slightly viscous | 94.86% |
| | | | | Screened by an |
| | | | | 80-mesh sieve |
| \textcircled{3} | 4451 | 44.51% | Slightly viscous | 97.13% |
| | | | | Screened by an |
| | | | | 80-mesh sieve |
| \textcircled{4} | 4328 | 43.28% | Slightly viscous | 96.30% |
| | | | | Screened by an |
| | | | | 80-mesh sieve |

| Spray-dried product | Angle of repose º | Fluidity determination |
|---|---|---|
| \textcircled{1} | 15.95° | Very good |
| \textcircled{2} | 8.88° | Very good |
| \textcircled{3} | 8.53° | Very good |
| \textcircled{4} | 11.31° | Very good |

| Spray-dried product | Weight gain rate | Hygroscopicity determination |
|---|---|---|
| \textcircled{1} | 36.13% | Hygroscopy |
| \textcircled{2} | 34.96% | Good hygroscopicity |
| \textcircled{3} | 34.69% | Good hygroscopicity |
| \textcircled{4} | 31.45% | Good hygroscopicity |

It can be known from the pilot spray drying state of the product that the fluidity and the hygroscopicity of the mixed extract after addition of a proper amount of Eucommia ulmoides Oliv. male flower are improved, and the yield is also increased. It should be emphasized that the mixed extract after addition of a proper amount of Eucommia ulmoides Oliv. male flower is easier to operate in the spray drying process, and the phenomenon of viscosity is reduced, so as to increase the yield of the material.

In combination with the results of the following pharmacodynamic experiments, the optimal proportion of the Eucommia ulmoides Oliv. leaf extract is: 80%-95% of the Eucommia ulmoides Oliv. leaves and 5%-20% of the Eucommia ulmoides Oliv. male flower. Preferably, the Eucommia ulmoides Oliv. leaves:the Eucommia ulmoides Oliv. male flower=90:10.

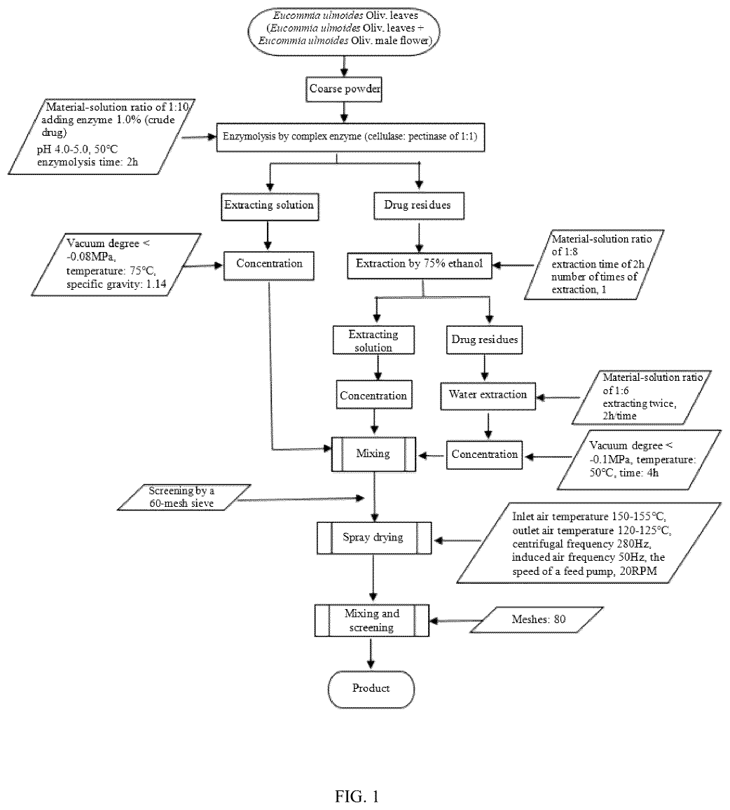

### 2.5 Technological Process is Shown in FIG. 1

### Embodiment 6

### Evaluation of Uric Acid Lowering Ability of Eucommia ulmoides Oliv. Leaves by Xanthine Oxidase (XOD) Inhibition

### I Method and Principle

Xanthine oxidase catalyzes a xanthine substrate to produce uric acid and superoxide anion, and uric acid has a characteristic absorption peak under 290 nm UV conditions. By measuring the change curve of absorbance value within a certain time, the slope of this curve is the production rate of the uric acid, to measure the change of the activity of xanthine oxidase, so as to judge the uric acid lowering ability of the sample.

### 1. Reagent

### 1.1 Phosphate Buffer Solution (PBS)

1.912 g of potassium dihydrogen phosphate moisture, 13.892 g of dipotassium hydrogen phosphate and 03724 g of EDTA(ethylenediamine tetraacetic acid) are weighed in a beaker, 300 ml of water is added for dissolving, the pH value is adjusted to 7.5, and the volume is fixed to 1000 mL.

1.2 Sodium Hydroxide Solution (1 mol/L)

4.0 g of sodium hydroxide is weighed in a 50 mL beaker and dissolved. After full dissolving and cooling to room temperature, the solution is transfer to a 100 mL volumetric flask with a glass rod for fixing the volume.

### 1.3 Xanthine Oxidase Solution

92.4 μL of xanthine oxidase mother solution (5 U/770 μL) is drawn in a centrifuge tube, and diluted with 11.9076 mL of PBS to obtain 0.05 U/mL xanthine oxidase working solution which shall be prepared for immediate use.

### 1.4 Xanthine Solution

0.02284 g of xanthine is weighed in a beaker; 1.2 mL of 1 mol/L NaOH solution is added; ultrasonic treatment is conducted for 20 min; and the volume is fixed to 100 mL with the PBS to obtain 1.5 mmol/L xanthine working solution.

1.5 Allopurinol/Eucommia ulmoides Oliv. Leaf Extract Solutions

Allopurinol: 0.0020 g of allopurinol is weighed; the volume is fixed to 100 mL with the PBS; the solution is thoroughly mixed well to prepare 20.0 mg/L mother solution; and the mother solution is diluted with the PBS to different multiples to obtain different concentrations of allopurinol solutions.

Eucommia ulmoides Oliv. leaf extract solutions: 0.50 g of corresponding Eucommia ulmoides Oliv. leaf extract sample in embodiment 5 is weighed; the volume is fixed to 50 mL with the PBS; the solution is thoroughly mixed well to prepare 10.0 mg/mL mother solution; and the mother solution is diluted with the PBS to different multiples to obtain different concentrations of Eucommia ulmoides Oliv. solutions.

1.6 Dilute Hydrochloric Acid Solution (1 mol/L)

8.33 ml of concentrated hydrochloric acid (12 mol/L) is drawn, and the volume is fixed to 100 mL with distilled water to prepare 1 mol/L dilute hydrochloric acid solution.

### 2. Experimental Steps

2.1 A proper quantity of centrifugal tubes are taken and numbered as OD1, OD2, OD3 and OD4 respectively. Reagents are added to each tube according to the combinations of Table 1.

TABLE 7
Addition Amount of Reagents

| Solution name | OD1 | | OD2 | | OD3 | | OD4 | |
|---|---|---|---|---|---|---|---|---|
| PBS | 0.5 | mL | 1.25 | mL | 1.0 | mL | 1.75 | mL |
| Xanthine oxidase | 0.5 | mL | 0.5 | mL | — | | — | |
| Allopurinol/Eucommia | 0.75 | mL | — | | 0.75 | mL | — | |
| ulmoides Oliv. leaf | | | | | | | | |
| extract | | | | | | | | |

Vortext oscillation and blending, 25° C. water bath, 15 min

| | | | | | | | | |
|---|---|---|---|---|---|---|---|---|
| Xanthine | 1.5 | mL | 1.5 | mL | 1.5 | mL | 1.5 | mL |

Vortex oscillation and blending, 25° C. water bath, 30 min

| | | | | | | | | |
|---|---|---|---|---|---|---|---|---|
| HCl | 1.0 | mL | 1.0 | mL | 1.0 | mL | 1.0 | mL |

Vortex oscillation and blending, detection OD_{290 nm}
Note:
the PBS solution is adjusted to zero and calibrated.

### 2.2 Experimental Data Processing

The concentrations are plotted in horizontal coordinates and the inhibition rates are plotted in vertical coordinates to calculate the concentration IC₅₀ with half inhibition rate.

$$XOD Inhibition rate ( \% ) = ( 1 - \frac{OD 1 - OD 3}{OD 2 - OD 4} ) \times 100 \%$$

In the formula:
OD1—refers to the absorbance value of an activity measurement system with the substrate, enzyme solution and inhibitor;
OD2—refers to the absorbance value of the activity measurement system with the substrate and enzyme solution and without allopurinol/sample;
OD3—refers to the absorbance value of the activity measurement system with the substrate and inhibitor and without enzyme solution;
OD4—refers to the absorbance value of the activity measurement system with the substrate and without allopurinol/sample and enzyme solution.

### 3. Experimental Results

### 3.1 In Vitro Inhibitory Effect of Allopurinol on Xanthine Oxidase

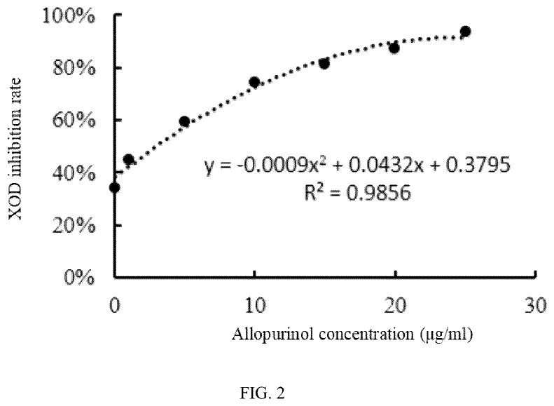

It can be seen from Table 8 and FIG. 2 that with the increase of the allopurinol concentration, the in vitro inhibitory activity of XOD is also enhanced, and the half inhibitory concentration IC₅₀ is 2.97 μg/mL.

TABLE 8
Inhibitory Effect of Allopurinol on Xanthine Oxidase

| Concentration | | | | | Inhibition |
|---|---|---|---|---|---|
| (μg/ml) | OD2 | OD4 | OD1 | OD3 | rate (%) |
| Control group | 2.262 | 0.241 | / | / | / |
| 0.05 | 2.262 | 0.241 | 1.563 | 0.231 | 34.09 |
| 1 | 2.262 | 0.241 | 1.354 | 0.241 | 44.93 |
| 5 | 2.262 | 0.241 | 1.064 | 0.244 | 59.43 |
| 10 | 2.262 | 0.241 | 0.765 | 0.24 | 74.02 |
| 15 | 2.262 | 0.241 | 0.627 | 0.248 | 81.25 |
| 20 | 2.262 | 0.241 | 0.507 | 0.249 | 87.23 |
| 25 | 2.262 | 0.241 | 0.385 | 0.25 | 93.32 |

3.2 In Vitro Inhibitory Effect of the Eucommia ulmoides Oliv. Leaf Extract on Xanthine Oxidase

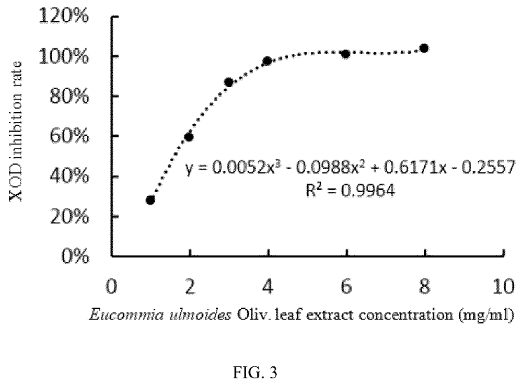

This experiment prepares Eucommia ulmoides Oliv. leaf extracts (test sample 1 in embodiment 5) with 6 concentration gradients of 1, 2, 3, 4, 6, and 8 mg/mL, and researches the inhibitory effects of the Eucommia ulmoides Oliv. leaf extracts with different concentrations on XOD. The experimental results are shown in Table 9 and FIG. 3. The Eucommia ulmoides Oliv. leaf extracts have certain inhibitory effects on XOD, and the half inhibitory concentration IC₅₀ is 1.60 mg/mL.

TABLE 9
Inhibitory Effect of Eucommia ulmoides Oliv. Leaf Extract
on Xanthine Oxidase

| Concentration | | | | | Inhibition |
|---|---|---|---|---|---|
| (mg/ml) | OD2 | OD4 | OD1 | OD3 | rate (%) |
| Control group | 2.246 | 0.246 | / | / | / |
| 1 | 2.246 | 0.246 | 2.468 | 1.027 | 27.95% |
| 2 | 2.246 | 0.246 | 2.589 | 1.777 | 59.40% |
| 3 | 2.246 | 0.246 | 2.585 | 2.318 | 86.65% |
| 4 | 2.246 | 0.246 | 2.628 | 2.577 | 97.45% |
| 6 | 2.246 | 0.246 | 2.883 | 2.903 | 101.00% |
| 8 | 2.246 | 0.246 | 2.928 | 3.001 | 103.65% |

3.3 In Vitro Inhibitory Effect of the Extracts with Different Eucommia ulmoides Oliv. Leaf Proportions on Xanthine Oxidase

This experiment is conducted once, prepares sample 1 to sample 4 and the Eucommia ulmoides Oliv. leaf extract with the concentration of 1.6 mg/mL in embodiment 5 respectively, and researches the inhibitory effects of different Eucommia ulmoides Oliv. leaf proportions on XOD. The experimental results are shown in Table 10. Different Eucommia ulmoides Oliv. leaf proportions play different effects, and different inhibitory effects on XOD. It can be known from the experiment that sample 2 has the best effect.

TABLE 10
Inhibitory Effects of Extracts with Different Ratios of Eucommia
ulmoides Oliv. Leaves and Eucommia ulmoides
Oliv. Male Flower on Xanthine Oxidase

| Concentration | | | | | Inhibition rate |
|---|---|---|---|---|---|
| (mg/ml) | OD2 | OD4 | OD1 | OD3 | (%) |
| Control group | 2.255 | 0.232 | / | / | / |
| Sample 1 | 2.255 | 0.232 | 2.630 | 1.584 | 48.29% |
| Sample 2 | 2.255 | 0.232 | 2.615 | 1.635 | 51.56% |
| Sample 3 | 2.255 | 0.232 | 2.623 | 1.618 | 50.32% |
| Sample 4 | 2.255 | 0.232 | 2.631 | 1.521 | 45.13% |

### Embodiment 7

### Experiment of Improvement Effect on Adenine Induced Chronic Renal Injury in Rats

### 1. Material and Method

### 1.1. Experimental Animals

Healthy male SD rats (140-160 g) without specific pathogen (SPF grade) are purchased from Sibeifu (Beijing) Biotechnology Co., Ltd. (SCXK (Jing) 2019-0010). Breeding environment: 23-25° C., relative humidity: 55%-60%, and illumination period, bright:dark is 12 h:12 h.

### 1.2 Chemicals and Samples

Adenine (high purity, 98%) is purchased from Shanghai Yuanye Bio-Technology Co., Ltd.

Test sample \textcircled{1} and sample \textcircled{2} are the samples in embodiment 5.

Malondialdehyde (MDA), neutrophil gelatinase-associated lipoprotein (NGAL), total antioxidant capacity (T-AOC) and a kit are purchased from Nanjing Jiancheng Bioengineering Institute, and IL-1β, TNF-α, complement C1q and CystatinC kit are purchased from Beijing Jingmei Biotechnology Co., Ltd.

### 1.3 Groups and Modeling

Groups:

| Groups | Drug | Dosage |
|---|---|---|
| Blank group | Normal saline | / |
| Model group | Adenine | 250 mg/kg bw · d⁻¹ |
| Control | Allopurinol positive drug | 25 mg/kg bw · d⁻¹ |
| group | | |
| Sample \textcircled{1} | Eucommia ulmoides Oliv. leaves: | 300 mg/kg bw · d⁻¹ |
| | Eucommia ulmoides Oliv. | |
| | male flower = 100:0 | |
| Sample \textcircled{2} | Eucommia ulmoides Oliv. leaves: | 300 mg/kg bw · d⁻¹ |
| | Eucommia ulmoides Oliv. | |
| | male flower = 90:10 | |

The SD rats (n=90) are placed in a room at temperature (22±3° C.) and humidity (65±5%) and with 12-hour dark/bright cycle for adaptation for 1 week. After the end of the adaptation period, except for the (9) rats in the normal control group, the other rats are fed with adenine by intragastric administration (250 mg/kg bw·d⁻¹) and provided with normal diet for 3 weeks to induce the establishment of a chronic kidney disease model. After 3 weeks of modeling, the successfully modeled rats are divided into 4 groups: a model control group (250 mg/kg bw·d⁻¹), an allopurinol positive drug group (25 mg/kg bw·d⁻¹), and test groups \textcircled{1} and \textcircled{2} (300 mg/kg bw·d⁻¹). At the same time, all groups are given normal food and water, and intragastric administration once a day (intragastric administration of normal saline is continued for the normal control group) for 8 weeks. The weight is measured once a week at the beginning of the test and after the test.

### 1.4 Sample Collection

After continuous intragastric administration for 8 weeks, all the rats are euthanized after fasting for 12 h. Blood is taken from the abdominal aorta, and centrifuged at 3000 r/min for 15 min, and serum is separated. The kidneys of the rats in the groups are separated, weighed and recorded. The removed right kidneys and part of the left kidneys are wrapped in tinfoil, soaked in liquid nitrogen and frozen at −80° C. for biochemical analysis. A small piece of the left kidney is placed in 4% formaldehyde fixative for subsequent histopathological examination. All serums and tissues are stored below −80° C. until use.

### 1.5 Detection of Serum Biochemical Indexes

The serum is obtained by centrifugation at 3500 r/min for 15 min. The levels of UR, UA and CR in the serum are determined by an automatic biochemical analyzer, and the levels of TNF-α, IL-β, MDA, complement C1q and CystatinC in the serum are detected by ELISA kit.

### 1.6 Histopathological Analysis of Kidney Tissues

The collected kidney tissues are soaked in 10% formalin for 2 d, then dehydrated with alcohol, fixed with paraffin, and cut into 5 μm thick slices. The slices are stained with hematoxylin (HE) for histopathological analysis. Immunohistochemical slices are incubated with osmotic solution and blocking buffer, incubated with target gene antibodies, treated with peroxidase conjugated streptavidin (Nichirei Co.) and 3,3-diaminobenzidine tetrahydrochloride (DBA reagent) successively, and finally stained with hematoxylin. The slices are dehydrated with fractional alcohol series, soaked in xylene twice and photomicrographed with a microscope.

### 1.7 Statistical Treatment

The data are expressed by “x±s”. One-way ANOVA is performed using SPSS software, and multiple comparisons are made between the groups. p<0.05 indicates significant difference, and p<0.01 indicates extremely significant difference.

### 2 Results

### 2.1 Determination Results of Serum and Urine Biochemical Indexes in Rats of Groups After Treatment

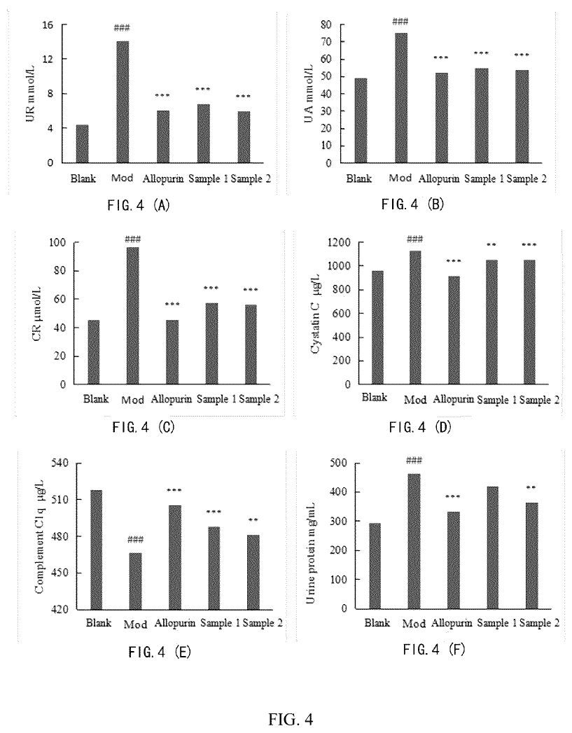

The determination results of serum biochemical indexes in rats of groups after treatment are shown in FIG. 4. As shown in the figure, compared with the blank group, the levels of UR, UA, CR, complement C1q, cystatin C and urine protein in the rats of the model group are significantly increased (p<0.001).

After intragastric administration of the test samples 1 and 2 for treatment, the UR changes are extremely significantly reversed (p<0.001).

After intragastric administration of the test samples 1 and 2 for treatment, the UA changes are extremely significantly reversed (p<0.001).

After intragastric administration of the test samples 1 and 2 for treatment, the CR changes are extremely significantly reversed (p<0.001).

After intragastric administration of the test sample 2 for treatment, the cystatin C changes are extremely significantly reversed (p<0.001), and after intragastric administration of the test sample 1 for treatment, the cystatin C changes are significantly reversed (p<0.01).

After intragastric administration of the test sample 1 for treatment, the complement C1q changes are extremely significantly reversed (p<0.001), and after intragastric administration of the test sample 2 for treatment, the complement C1q changes are significantly reversed (p<0.01).

After intragastric administration of the test sample 2 for treatment, the urine protein changes are significantly reversed (p<0.001).

After treatment, the determination results of the serum biochemical indexes of the rats in each group show that when the ratio of the Eucommia ulmoides Oliv. leaves to the Eucommia ulmoides Oliv. male flower is 90:10 in the test drug group 2, the effects of the detection indexes are the best.

Wherein all data in FIG. 4 are expressed as mean±standard deviation. *p<0.05, **p<0.01, ***p<0.001, compared with the Mod group; ###p<0.001, compared with the Con group.

### 2.2 Detection Results of Antioxidant Index and Inflammatory Factor of Rats of Groups After Treatment

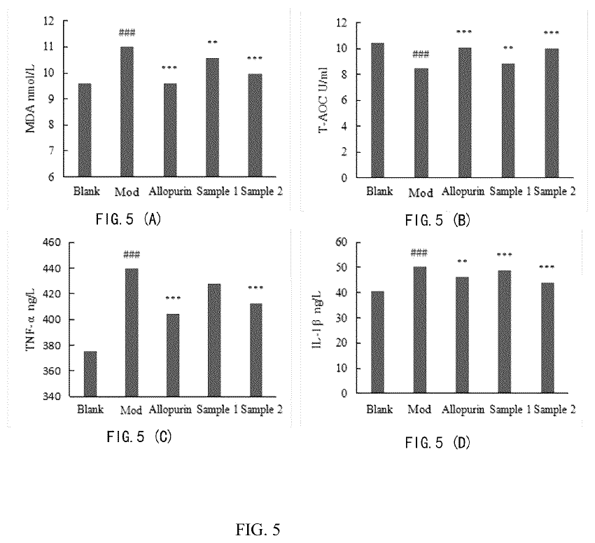

The detection results of the antioxidant index and the inflammatory factor of the rats of groups after treatment are shown in FIG. 5. After treatment, compared with the Con group, the level of T-AOC of the rats in the Mod group are extremely significantly decreased (p<0.001), and MDA, TNF-α and IL-β are extremely significantly increased (p<0.001).

After intragastric administration of the test drug group for treatment, compared with the model group, the level of MDA of the test drug group 2 is extremely significantly increased (p<0.001) and the level of MDA of the test drug group 1 is significantly increased (p<0.01).

After intragastric administration of the test drug group for treatment, compared with the model group, the level of T-AOC of the test drug group 2 is extremely significantly increased (p<0.001) and the level of T-AOC of the test drug group 1 is significantly increased (p<0.01).

After intragastric administration of the test drug group for treatment, compared with the model group, the level of TNF-α of the test drug group 2 is extremely significantly decreased (p<0.001).

After intragastric administration of the test drug group for treatment, compared with the model group, the levels of IL-β of the test drug groups 1 and 2 are extremely significantly decreased (p<0.001).

The detection results of the antioxidant index and the inflammatory factor of the rats of groups after treatment show that when the ratio of the Eucommia ulmoides Oliv. leaves to the Eucommia ulmoides Oliv. male flower is 90:10 in the test drug group 2, the effects of the detection indexes are the best.

Wherein all data in FIG. 5 are expressed as mean±standard deviation. *p<0.05, **p<0.01, ***p<0.001, compared with the Mod group; ###p<0.001, compared with the Con group.

### 2.5 Histopathological Analysis of Kidney Tissues

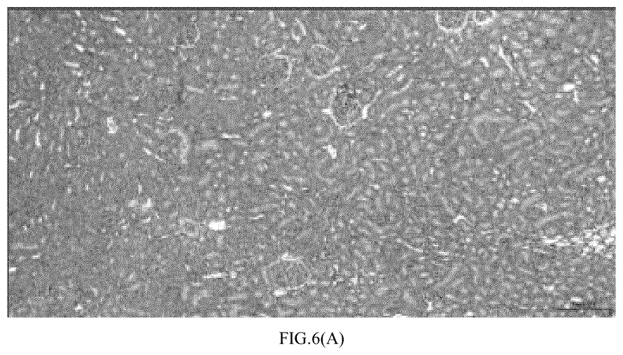

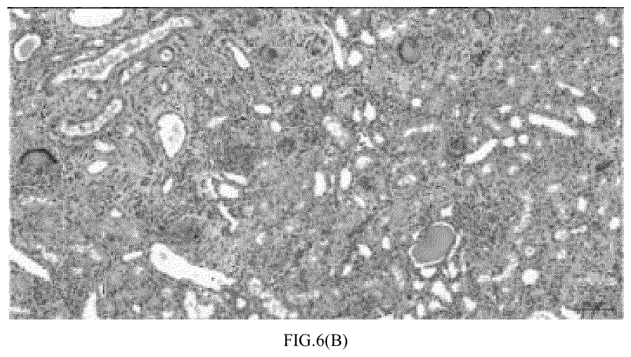

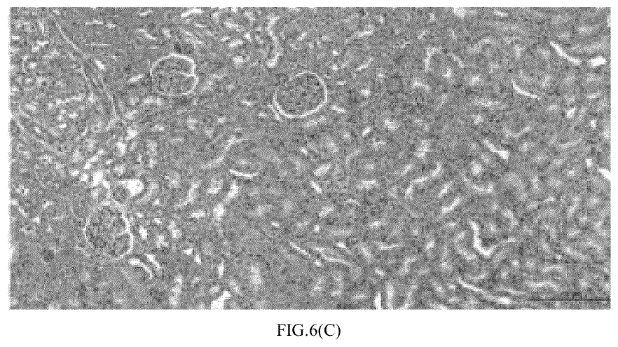

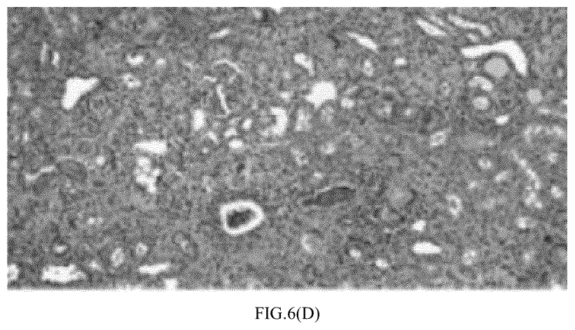

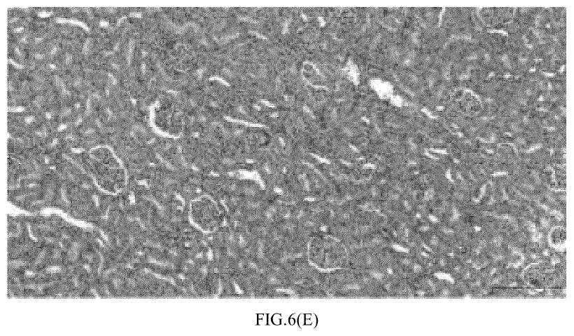

The influence of the Eucommia ulmoides Oliv. leaf extract (sample 1 and sample 2 in embodiment 4) on renal histopathology in rats is shown in FIG. 6. Compared with the normal group, the brown crystal deposition in the renal tubule interstitium, great expansion of renal tubules, mesangial hyperplasia and atrophy of glomerulus and much inflammatory cell infiltration in the glomerulus can be observed in the kidney tissue of the rats in the model group under a light microscope. Compared with the model group, the above pathological changes in sample 1 and sample 2 are alleviated.

In conclusion, the Eucommia ulmoides Oliv. leaf extract can protect the kidney structure and reduce the levels of serum UA, CR, UR, cystatin C, complement C1q and urine protein in urine, to improve CKD. The mechanism may be related to the enhancement of antioxidant capacity and anti-inflammation.

### Embodiment 8

### Quality Control

I Fingerprint Detection Method for Eucommia ulmoides Oliv. Leaf Extract

### 1. Instruments and Equipment

High performance liquid chromatography Daojin LC-20A

Electronic scales Hundred thousandth analytical scales (METTLER TOLEDO MS105DU)

### 2. Reagent and Material

Acetonitrile (Fisher chromatography pure), water (Watsons, distilled water), phosphoric acid (Chem, chromatography grade); microporous filter membrane (BOJIN nylon 0.22 μm), and syringe (Jiangxi Qingshantang Medical Equipment, 1 mL)

### 3. Reference Chromatographic Conditions

Daojin Shim-pack VP-ODS (4.6×250 mm, 5 μm)

### 4. Chromatographic Conditions and System Suitability Test

Gradient elution is performed as specified in the table below by taking octadecyl silane chemically bonded to porous silica as the filling agent, 0.4% phosphoric acid solution as mobile phase A and acetonitrile s mobile phase B. Detection wavelength is 254 nm; the flow rate is 1.0 mL/min; and column temperature is 35° C.

| Time (min) | Mobile phase A (%) | Mobile phase B (%) |
|---|---|---|
| 0 | 95 | 5 |
| 10 | 90 | 10 |
| 30 | 88 | 12 |
| 65 | 70 | 30 |
| 85 | 5 | 95 |
| 86 | 95 | 5 |

### 5. Preparation of Reference Substances and Test Substances

0.5 g of Eucommia ulmoides Oliv. leaf extract (sample 2 in embodiment 3) is accurately weighed in a 100 ml conical flask, about 25 mL of 50% methanol is added, ultrasonic treatment is conducted for 30 min, and the solution is cooled to room temperature, shaken well, and filtered to obtain the test solution.

Various reference substances (geniposidic acid, catechuic acid, protocatechuic aldehyde, syringin, chlorogenic acid, asperuloside, rutin, isoquercitrin, kaempferol 3-rutinoside, quercitrin, quercetin and kaempferol) are prepared into 1 mg/mL reference solutions with methanol respectively, and 1 mL of the reference solutions is taken, blended respectively and filtered to obtain the mixed reference solution.

### 6. Determination Method

10 μL of the test solution and mixed reference solution is accurately drawn and injected into the liquid chromatograph for determination.

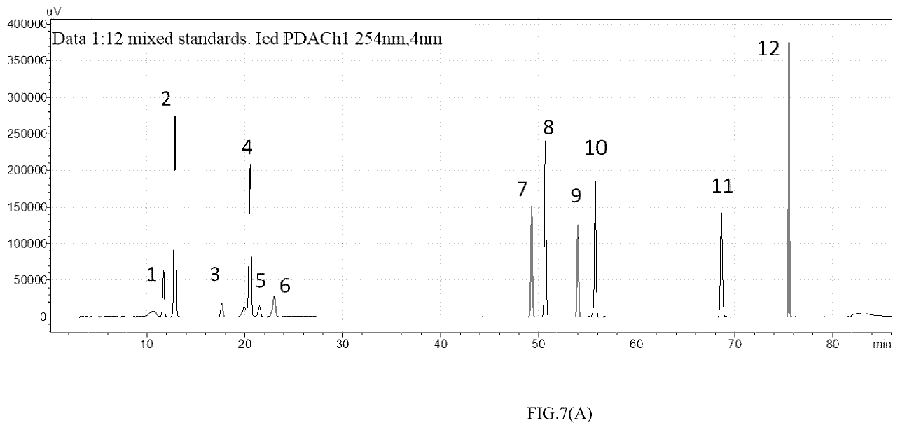

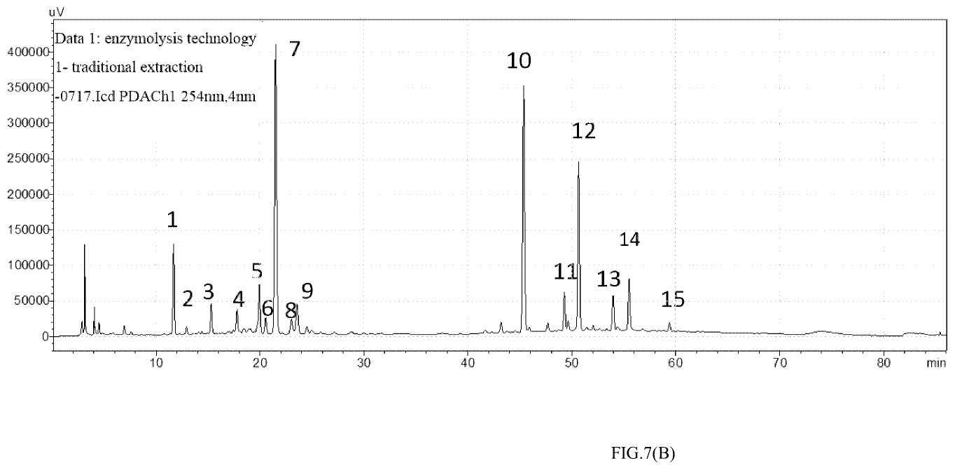

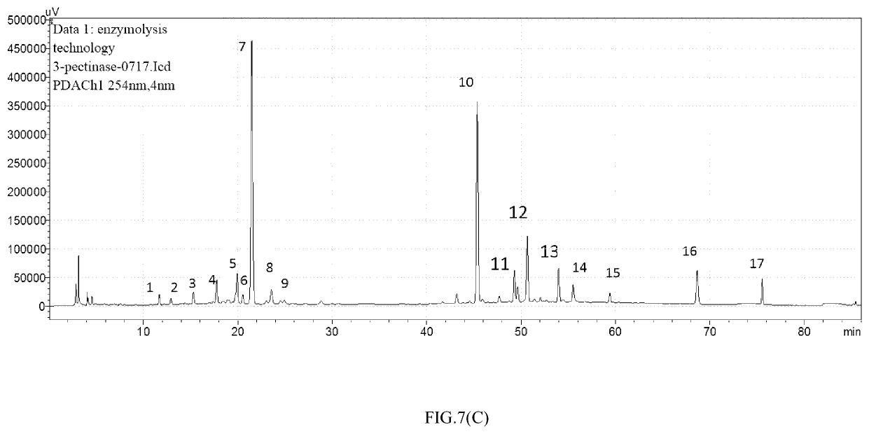

7. The chromatogram of the Eucommia ulmoides Oliv. leaf composite extract is shown in FIG. 7.
wherein A is a control standard, B is a sample directly extracted without

enzymolysis process, and C is a Eucommia ulmoides Oliv. leaf sample after enzymolysis.

In A, peak 1: geniposidic acid; peak 2: catechuic acid; peak 3: protocatechuic aldehyde; peak 4: syringin; peak 5: chlorogenic acid; peak 6: asperuloside; peak 7: rutin; peak 8: isoquercitrin; peak 9: kaempferol 3-rutinoside; peak 10: quercitrin; peak 11: quercetin; peak 12: kaempferol;

In B, peak 1: geniposidic acid; peak 2: catechuic acid; peak 3: neochlorogenic acid; peak 4: protocatechuic aldehyde; peak 6: syringin; peak 7: chlorogenic acid (S) peak 8: asperuloside; peak 9: cryptochlorogenic acid; peak 11: rutin; peak 12: isoquercitrin; peak 13: kaempferol 3-rutinoside; peak 14: quercitrin;

In C, peak 1: geniposidic acid; peak 2: catechuic acid; peak 3: neochlorogenic acid; peak 4: protocatechuic aldehyde; peak 6: syringin; peak 7: chlorogenic acid (S) peak 8: asperuloside; peak 9: cryptochlorogenic acid; peak 11: rutin; peak 12: isoquercitrin; peak 13: kaempferol 3-rutinoside; peak 14: quercitrin; peak 16: quercetin; peak 17: kaempferol.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. A preparation method for a Eucommia ulmoides Oliv. leaf extract, specifically comprising the following steps:
1) soaking the coarse powder of Eucommia ulmoides Oliv. leaves or mixed coarse powder of Eucommia ulmoides Oliv. leaves and male flower in water, and then adding a complex enzyme for enzymolysis and filtration to obtain drug residues and extracting solution I;
2) adding ethanol to the drug residues prepared in step 1) for extracting for 1-2 times; and after screening and filtering, obtaining drug residues and extracting solution II;
3) adding water to the drug residues prepared in step 2), and after extracting for 1-2 times, filtering to obtain extracting solution III;
4) concentrating the extracting solution I, the extracting solution II and the extracting solution III respectively; cooling the concentrates to room temperature and then mixing the concentrates; standing and then filtering; and spray-drying the filtrate to obtain the Eucommia ulmoides Oliv. leaf extract.

2. The preparation method for the Eucommia ulmoides Oliv. leaf extract according to claim 1, wherein in step 1), the material-solution ratio of the coarse powder or mixed coarse powder of the Eucommia ulmoides Oliv. leaves to the water is 1:10-20, and the complex enzyme is a mixture of cellulase and pectinase with a mass ratio of 1:1-3:1; the addition amount of the complex enzyme is 1%-2% of the mass of crude drug, enzymolysis time is 1-3 hours, and enzymolysis temperature is 45° C.-50° C.

3. The preparation method for the Eucommia ulmoides Oliv. leaf extract according to claim 1, wherein in step 2), the material-solution ratio of the drug residues to the ethanol is 1:6-10, the ethanol concentration is 70%-80%, and the extraction time is 1-3 hours each time.

4. The preparation method for the Eucommia ulmoides Oliv. leaf extract according to claim 1, wherein in step 3), the material-solution ratio of the drug residues to the water is 1:6-1:8, and the extraction time is 1-2 hours each time.

5. The preparation method for the Eucommia ulmoides Oliv. leaf extract according to claim 1, wherein the concentration technology in the step 4) is rotary evaporation with a vacuum degree of −0.06 to −1 Mpa, temperature of 60-80° C. and speed of 40-60 rpm; the density of the extract at 25° C. is 1.12-1.14; and the technological parameters for centrifugal spray drying of the obtained concentrates are as follows:
inlet air temperature is 150-155° C., outlet air temperature is 120-125° C., the centrifugal frequency is 280 Hz, induced air frequency is 50 Hz, and the speed of a feed pump is 20 r/min.

6. A Eucommia ulmoides Oliv. leaf extract prepared by the method of claim 1, wherein the Eucommia ulmoides Oliv. leaf extract is obtained from the Eucommia ulmoides Oliv. leaves or the mixture of the Eucommia ulmoides Oliv. leaves and the Eucommia ulmoides Oliv. male flower by enzymolysis and complex extraction of ethanol and water; and the mass percentages of the Eucommia ulmoides Oliv. leaves and the Eucommia ulmoides Oliv. male flower in the mixture are as follows:
80%-95% of the Eucommia ulmoides Oliv.leaves and 5%-20% of the Eucommia ulmoides Oliv .male flower;
and the Eucommia ulmoides Oliv. leaf extract has the effect of protecting the health of the kidney function.

7. The Eucommia ulmoides Oliv. leaf extract according to claim 1, wherein the mass ratio of the Eucommia ulmoides Oliv. leaves to the Eucommia ulmoides Oliv. male flower in the mixture is 90:10.

8. A fingerprint detection method for the Eucommia ulmoides Oliv. leaf extract prepared by the method of claim 1, wherein high performance liquid chromatography is used for constructing a chromatogram, and chromatographic conditions are as follows:
chromatographic columns use octadecyl silane chemically bonded to porous silica as fillers; column temperature is 25-35° C.; a flow rate is 0.9-1.1 mL/min; a detection wavelength is 203-337 nm; a mobile phase A is 0.4% phosphate water, and a mobile phase B is acetonitrile; and a gradient elution procedure is:

| Time (min) | Mobile phase A (%) | Mobile phase B (%) |
|---|---|---|
| 0 | 95 | 5 |
| 10 | 90 | 10 |
| 30 | 88 | 12 |
| 65 | 70 | 30 |
| 85 | 5 | 95 |
| 86 | 95 | 5 |

9. The fingerprint detection method for the Eucommia ulmoides Oliv. leaf extract according to claim 1, wherein a preparation method for a test solution used for liquid chromatography determination in the method comprises:
accurately weighing 0.5 g of Eucommia ulmoides Oliv. leaf extract in a 100 ml conical flask, adding 25 mL of 50% methanol, conducting ultrasonic treatment for 30 min, cooling to room temperature, shaking well, and filtering to obtain the test solution;
a preparation method for a reference solution comprises: preparing various reference substances used for fingerprint detection into 1 mg/mL reference solutions with methanol respectively, blending 1 mL respectively and filtering to obtain the mixed reference solution.
